(19) United States
(12) Patent Application Publication
(10) Pub. No.: US 2008/0015460 A1
(43) Pub. Date: Jan. 17, 2008

(54) Adaptive Data Analysis

(76) Inventors: Alexander Kanevsky, Arad (IL); Ilia Kreiman, Beer Sheva (IL)

Correspondence Address: NATH & ASSOCIATES, 112 South West Street, Alexandria, VA, 22314, US

(21) Appl. No.: 11/628,113

(22) PCT Filed:
(86) PCT No.: PCT/IL05/00546

(30) Foreign Application Priority Data
Jun. 2, 2004 (IL) 162312

(51) Int. Cl. A61B 5/053

(52) U.S. Cl. 600/547

(57) ABSTRACT

A diagnostic method and device to which the data analysis method of the invention is applied comprises selecting a number of biologically active points (BAPs), measuring the skin resistance at each one of the points relative to two fixed resistance values corresponding to a lower border and to an upper border of skin resistances, without stimulation and after stimulation, whereby to obtain two sets of measurement results, a first set for non-stimulated BAPs and a second set for the same BAPs after being stimulated, for each set calculating the average resistance for these points as a first and a second isoelectric line, respectively, for which a first and a second normal corridors are respectively defined.

### FIELD OF THE INVENTION

The present invention relates to a method for non-invasive diagnosis of actual and potential disease activity. More particularly, the present invention relates to a non-invasive diagnosis procedure that adapts itself to the diagnosed person.

### BACKGROUND OF THE INVENTION

It has been known for some time that there are points in the skin of the human body in which the electric conductivity is higher than in the surrounding area, as a result of some actual or potential pathological phenomena. It has also been found that each one of these points is related to a particular organ of the body. All the points that are associated with the same organ are arranged in a line known in the field as a meridian.

The points of the body at which the electrical resistance is measured on the skin have been called BAPs (Biologically Active Points). It has been found that for each meridian there is one BAP that represents that meridian and provides an average value therefor.

In WO 01/56461, the inventor of the current invention describes in detail a method for utilizing source-points, announcement points, sympathetic points and energy reference points for assessing the physiological condition of a diagnosed person. Briefly, according to WO 01/56461, 24 BAPs are selected and the skin resistance at said points is measured twice to form two sets of results. The first set of measurement results includes the skin resistance at said 24 BAPs without stimulating these points, whereas the second set comprises measurement results of skin resistance at the same 24 BAPs after stimulating. these points. Then, a normal corridor is conventionally used (sometimes referred to hereinafter as a universal corridor), and, according to WO 01/56461, if both a specific result from the first set of measurements (i.e., before applying stimulation to the BAPs) and a corresponding result from the second set of measurements (i.e., after stimulating the BAPs) fall outside the normal corridor, these two specific results indicate the presence of a disease in the related organ. However, if one of the results in one of the two sets of measurements falls inside the normal, or universal, corridor, then the corresponding result from the other set of measurements, if it falls outside the universal corridor, is considered a false disease indication and is therefore disregarded. In other words, if there is a measurement result, either from the first or from the second set of measurements, that exceeds the universal corridor (hereinafter referred to as a meaningful result), which can potentially indicate an infected organ, this meaningful result might be rendered useless by a corresponding result from the other set of measurements should the latter result lays within the universal corridor.

A measurement result that lies within a universal corridor is referred to hereinafter as concealed result.

An attempt to utilize the concealed result might lead to erroneous diagnostics of the related organ and, therefore, the meaningful result is usually disregarded, whereby to waste the time, money and instrumentation involved in the measurement procedure. The aforesaid problem (of disregarding meaningful results) is due to the fact that the normal corridor is utilized irrespective of the actual BAP measurements of the diagnosed person. Since different persons have different physiological and mental characteristics, utilization of a universal corridor irrespectively of the diagnosed persons might lead, in many cases, to results being concealed, and, thus, to meaningful results being useless.

According to WO 01/56461, two sets of measurements are said to be compared, e.g., by superimposing them on one another on, e.g., a computer display screen, and diagnostic conclusions are reached based on the comparison. However, in some cases, one or more of the measured values, which can belong to the first, second or both sets of measurements, become concealed after being superimposed on one another, because the “concealed” measurement resides entirely within the universal corridor. In such cases, no decisive medical decision can be made with respect to the organ whose BAP measurement value is concealed. Therefore, it would be beneficial, in such cases, to modify the corridor such as to make concealed measurements available to the therapist, to allow him to consider every measurement and, thus, to obtain more accurate conclusions regarding problematic organs of the monitored person.

It is therefore an object of the present invention to provide a non-invasive method for disease diagnosis, according to which the normal corridor is normalized to obtain a decisive decision as to physiological condition of a diagnosed person.

It is another object of the present invention to provide a diagnosis procedure that is optimized to the diagnosed person.

Other objects and advantages of the invention will become apparent as the description proceeds.

### SUMMARY OF THE INVENTION

The term normal corridor (the terms normal and universal being interchangeably used hereinafter) is meant hereinafter as the corridor referred to in WO 01/56461, the whole specification of which is incorporated herein by reference. According to WO 01/56461, Nakatani's normal corridor, which is a corridor relating to a current span of about 2.50 microamperes, is used for diagnosis.

The term BAPs of interest is meant hereinafter as BAPs that belong to one or more meridians relating to one or more organs of a patient, the physiological condition of which is sought.

The present invention provides an improved data analysis method, useful in a non-invasive diagnostic method for disease diagnosis, according to which the normal corridor is, whenever required—as described hereinafter—modified, to optimize a diagnostic procedure to a monitored person in the way described hereinafter.

The diagnostic method to which the data analysis method of the invention is applied comprises selecting X biologically active points (BAPs), measuring the skin resistance at each one of said points relative to two fixed resistance values corresponding to a lower border and to an upper border of skin resistances, without stimulation and after stimulation, whereby to obtain two sets of measurement results, a first set for non-stimulated BAPs and a second set for the same BAPs after being stimulated, for each set calculating the average resistance for these points as a first and a second isoelectric line, respectively, for which a first and a second normal corridors are defined, respectively, the method being:
(a) if the value of the first and/or second isoelectric line, as the case may be, falls between the values of said lower border and said higher border, the corresponding corridor(s) remains unmodified, whereas if the value of the first and/or second isoelectric line falls below the value of said lower border, the corresponding normal corridor(s) is modified to have a narrower width, or if the value of the first and/or second isoelectric line is greater than the value of said upper border, the corresponding normal corridor(s) is modified to have a larger width, the resulting modified width depends on the relationship existing between the value of a specific isoelectric line to the values of said lower and upper borders; and
(b) diagnostic conclusions are reached based on the measurement results before and after the stimulation, and on the first and/or second normal corridors, whether modified or unmodified, whichever the case maybe. According to the invention, the diagnostic conclusions obtainable through the improved data analysis comprise the following:
(a) If a measurement result belonging to the first set of measurement results falls outside the first modified, or unmodified, corridor, this result is considered a potential indicator of disease activity; and
(b) If the corresponding result belonging to the second set of measurement results also falls outside the second modified, or unmodified, corridor, the potential indicator is considered as a true indication of the presence of a disease; otherwise, said potential indicator is a false indicator; that is, the indicator result is considered not to be an indication of a true disease state and is, therefore, disregarded.

According to the invention, the modification of the width of the first and/or second normal corridors is performed by:
(a) taking a first set of results previously obtained by selecting one or more BAPs of interest and measuring the skin resistance at these points without stimulating them, whereby to obtain a first set of resistance results. The first set of results comprises the group of measurement[i], where i=1, 2, 3, . . . , (up to i=X);
(b) calculating the isoelectric line (AV1) relating to the first group of measurements measurement[i] (i =1, 2, 3, . . . , X):

$$AV 1 = ( {\sum }_{i = 1}^{x} measure [ i ] ) / X$$

where ‘X’ is the number of the measured BAPs;
(c) modifying the universal corridor to obtain the corridor (I_{MC1}) relating to the first isoelectric line (AV1) using the following rules:
(c.1) if AV1<I2, then:

$${I}_{MC 1} = \frac{AV 1}{I 2} \times In ( in μ A )$$

I2 (the lower border of a corridor with respect to any isoelectric line) equals: I2=U/(R_{dev}+Rᵤₚ)−In(in μA),
‘U’ is the magnitude (in volts) of the voltage source,
‘R_{dev}’ is the intrinsic (i.e., “self”) resistance of the measurement apparatus (in kΩ), and
‘Rᵤₚ’ designates the highest value (in kΩ) of a range, the lower value of which is designated by ‘R_{low}’ (in kΩ), and the electrical resistance of BAPs normally (i.e., statistically) residing within the range {R_{low}, Rᵤₚ};
(c.2) if I2≦AV1≦I1, then
   I_{MC1}=In(in μA)
   where I1 is the upper border of the isoelectric line and equals: I1=U/(R_{dev}+R_{low}) (in μA), and ‘In’ designates the span of the normal corridor (in μA); and
(c.3) if AV1>I1, then

$${I}_{MC 1} = \frac{AV 1}{I 1} \times In ( in μ A )$$
(d) taking a second set of results previously obtained by applying stimulation to the BAPs used in step (a), and, essentially immediately thereafter, measuring the skin resistance at said points, whereby to obtain a second set of resistance results. The second set of results comprises the group of measurement[i], where i=1′, 2′, 3′, . . . , (up to X BAPs, for which i=X′), where the indexes 1′ and 1 refer to the same BAP (1′—after stimulation, and 1—before stimulation), and so on;
(e) calculating a second isoelectric line (AV2) relating to the second group of measurement.[i], (i=1′, 2′, 3′, . . . , where 1 and 1′ refer to the same BAP, and so on):

$$AV 2 = ( {\sum }_{i = {1}^{′}}^{x} measure [ i ] ) / X$$
(f) modifying the universal corridor to obtain the corridor (I_{MC2}) relating to the second isoelectric line (AV2) using the following rules:
(f.1) if AV2<I2, then:

$${I}_{MC 2} = \frac{AV 2}{I 2} \times In ( in μ A )$$
(f.2) if I2≦AV2≦I1, then
   I_{MC2}=In(in μA)
(f.3) if AV2>I1, then

$${I}_{MC 2} = \frac{AV 2}{I 1} \times In ( in μ A )$$

According to an aspect of the invention, an average diagram is plotted, upon which measurement results of the first and second sets are superimposed, after normalization and modification (if relevant), and compared.

The normalization and superposition are performed by:
(a) Calculating a first difference Δ1=|I_{MC1}−I_{MC2}| and a second difference Δ2=|AV1−AV2|;
(b) modifying the group of measurements measurement[i] to obtain a modified set of results measurement[i]_{modified}:
   measurement[i]_{modified}=measurement[i]+Δ1/2+Δ2
   where,
   if I_{MC1}<I_{MC2}, then i=1, 2, 3, . . . , (up to X), or
   if I_{MC1}>I_{MC2}, then i=1′, 2′, 3′, . . . , (up to X′); and
(c) superimposing the (first or second) group of measurements, after modifying it in step (b) above, on the unmodified (second or first) group of measurements, and plotting the results on a same diagram for comparison. It is noted that it may occur that the modification factor will be zero (i.e., Δ1/2+Δ2=0), in which case no actual modification will occur (i.e., measurement[i]_{modified}=measurement[i]).

In one embodiment of the present invention, the number (A) of the BAPs is 24.

According to another aspect of the present invention, there is provided a device adapted to carry out the diagnostic method and related calculations as detailed above, including carrying out measurements of the BAPs, transforming their results into numerical data, and transmitting the data to a separate processing unit, such as a computer. The device applies a consistent pressure to all BAPs to be measured. This pressure may be about 0.5 Dj/cm². The device may further be adapted to provide the stimulation.

The device is adapted to take several measurements of each BAP within a relatively short time, e.g., 5 measurements in 0.02 seconds. The device calculates the range of measured values. If the range is more than a predetermined amount, e.g., 5%, then the measurements are repeated until such time that all of the measurements taken are within the range. Each point is ideally not measured for more than a certain amount of time, e.g., 0.2 seconds.

By using a consistent pressure and ensuring that all measurements taken are within a predetermined range of values, the accuracy of the device is improved.

A description regarding the names of the usually used BAPs, the way in which the conductivity of the BAPs is measured, and how additional, complementary, points (e.g., announcement and sympathetic points) are used either to confirm or question the resulting information that is obtained using the principles of this invention, is included in WO 01/56461.

### DETAILED DESCRIPTION OF EMBODIMENTS

The principles of the invention will be now demonstrated by way of an example.

It has been statistically found, that the electrical resistance of BAPs is characterized by being within the range of 230 to 250 kΩ. This range is utilized in the invention to normalize resistances of BAPs of interest.

### Example

### Diagnosing a Disease Related to a Human Liver

The voltage source (U) that was used for stimulation of the BAPs had a magnitude of 5 VDC. In addition, the electrical resistance of the measurement equipment (R_{device}, also denoted herein by R_{dev}) was 250 kΩ, and the electrical current (I), which was indicative of the electrical resistance R of the skin at the monitored points (BAPs), was calculated using the formula I=U/(R_{dev}+R).

As noted hereinbefore, the universal Nakatani corridor is known in the art to have a fixed current span (‘In’), or width, of 2.5 μA (In=2.5 μA), which is conventionally used irrespective of the measurement results of BAPs.

As known in the art, the normative, or universal, corridor is superimposed on what is commonly referred to in the art as an “isoelectric line,” which refers to a current value that represents the average of a plurality of current measurements relating to the monitored BAPs. The normative corridor is superimposed on the isoelectric line such that the upper gap, which is the gap between the upper border of the corridor and the isoelectric line, equals to the lower gap, which is the gap between the lower border of the corridor and the isoelectric line. In other words, the equal gaps have, in the case of a universal corridor, fixed values: ±1.25 μA above and below the isoelectric line. The resistance of the BAPs was measured before and after stimulation by use of the measuring way described in WO 01/56461.

As noted hereinbefore, the present invention is characterized in that the normative, or universal, corridor is modified whenever a particular measurement of a specific BAP, which relates to a human organ of interest, is “concealed” by the universal corridor. An exemplary modification of the universal corridor is described in detail hereinafter.

In this example, the resistance of 24 BAPs (X=24) points was measured before and after stimulation, to obtain two sets of 24 measurements—a first set of 24 measurements before stimulation, and a second first set of 24 measurements after stimulation of the same 24 BAPs. Then, each set of 24 measurements was averaged. The average value (AV1) of the 24 measurements before the stimulation was calculated to be AV1=6.00 μA. Accordingly, the isoelectric line, which represents the 24 measurement values, equals AV1=6.00 μA.

According to the prior art, the universal corridor (In=2.50 μA) is located between 7.25 μA (i.e., AV1+2.50/2=6.0+1.25) and 4.75 μA (AV+2.50/2=6.0−1.25), as shown in Table 1. Because the example set forth refers to the diagnosis of a human liver, a particular attention is given to the measurement corresponding to the BAP of the liver of the monitored person, which was found to equal to 7.15 μA, as also shown in Table 1.

TABLE 1
(1^{st}, original set of measurement results before stimulation)

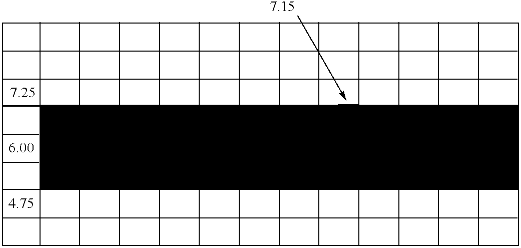

As shown in Table 1, the measured value 7.15 μA, which corresponds to the liver of the diagnosed person (denoted by ‘L’ in Table 1), does not exceed the universal corridor 4.75 to 7.25 μA, which means that probably there is no deviation from the normal functioning of the physiological system relating to the liver.

The 24 stimulated BAPs were also averaged, and the average value AV2 was calculated to be 7.70 μA (AV2=7.70 μA). According to the prior art, the universal corridor (In=2.50 μA) is to be located between 8.95 μA (i.e., AV2+2.50/2=7.70+1.25) and 6.45 μA (AV2+2.50/2=7.70−1.25), as shown in Table 2. It was found that the measurement corresponding to the stimulated BAP of the liver was equal to 10.00 μA, as also shown in Table 2.

TABLE 2
(2^{nd}, original, set of measurement results after stimulation)

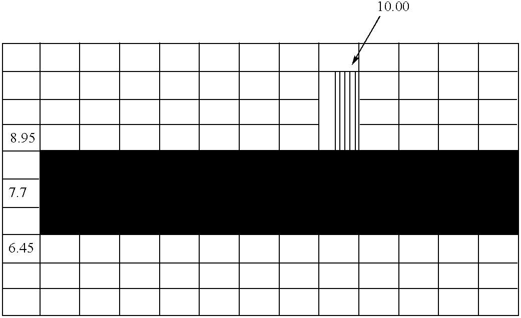

The measurement result relating to the liver (Table 2) exceeds, what is regarded by those skilled in the art as, the normal activity of the liver physiological system (L), which might indicate a problematic liver. However, according to Table 1 the measurement result relating to the BAP before applying the stimulation does not exceed the normal activity value that relates to the normal functioning of the liver; i.e., this measurement result (shown in Table 1) is “concealed,” or “hidden,” by the universal corridor. Therefore, no decisive conclusion can be obtained from the two sets of 24 measurements, regarding the physiological condition of the diagnosed liver, which is based solely on the measurements shown in Tables 1 and 2.

The results shown in Tables 1 and 2 are superimposed on one another, and the result is shown in Table 3:

TABLE 3
(1^{st}, original set conventionally superimposed on 2^{nd} original set)

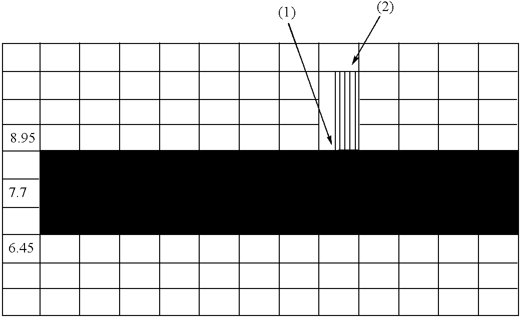

After being superimposed on one another, as shown in Table 3, the first measurement result (marked as ‘(1)’) of the BAP relating to the liver (marked as ‘L’) is shown residing completely in the universal corridor, the lower and upper borders of which are 6.45 and 8.95 μA, respectively, and, therefore, one cannot decisively conclude whether the liver is indeed problematic or not.

Table 3 demonstrates the conventional approach and a common situation, according to which measurement results that relate to infected organs (e.g., Liver), may fall inside the universal corridor and, therefore, they will be disregarded for failing to indicate probable problematic organs.

A different problem of the conventional approach is that sometimes measurement results, which relate to healthy organs, may fall outside the normal corridor, in which case they will be erroneously considered as indications for infected organs.

In order for the therapist to overcome the above-described problems and to be able to reach a decisive conclusion as to the physiological condition of, e.g., the liver, while utilizing the two originally obtained sets of 24 measurement results, the universal corridor is modified/normalized, for the first set of 24 measurement results, or for the second set of measurement results, or both for the first and for the second sets of measurement results, as the case may be in the following way:

Assuming that BAPs normally have an electrical resistance within the practical range of 225 kΩ to 255 kΩ—which is derived from the above-noted 230-250 kΩ and while considering deviations of about 2% of the intrinsic resistance of the measuring equipment—and that the exemplary voltage source is U=5 VDC, the upper border of the isoelectric line I1 is calculated using the lowermost value of the resistance range (i.e., R_{low}=225 kΩ):
I1=U/(R_{dev}+R_{low})=5/(250+225)=10.6 μA
whereas the isoelectric line (I2) is calculated using the higher most value of the resistance range (i.e., Rᵤₚ=255 kΩ) and the normative corridor (i.e. In=2.50 μA):
I2=U/(R_{device}+Rᵤₚ)−In=5/(250+255)−In=9.9−2.5=7.4 μA
Then, the following calculations are performed utilizing the latter calculated I1 and I2 (i.e., I1=10.60 μA , and I2=7.40 μA):
1. A modified corridor (I_{MC1}) is found for the first set of 24 measurement results (i.e., before applying stimulation), as follow:

$${I}_{MC 1} = \{ \begin{matrix}( AV 1 / I 2 ) \times In ; & if AV 1 < I 2 \\ In ; & if I 2 \leq AV 1 \leq I 1 \\ ( AV 1 / I 1 ) \times In ; & if AV 1 > I 1\end{matrix}$$

For the first set of 24 measurements the condition AV1<I2 is met (i.e., AV1=6.00<I2=7.40), and, therefore, the first modified corridor (I_{MC1}) is:
I_{MCl}=(A1/I2)×In=(6.00/7.40)×2.50=2.0 μA

Accordingly, the upper border of the modified corridor coincides with the 7.0 μA line, whereas the lower border of the modified corridor coincides with the 5.0 μA line, as shown in Table 4. Referring again to the BAP relating to the liver, the original measurement result thereof before the stimulation (i.e., 7.15 μA) is shown in Table 4 falling outside the modified (now narrower) corridor (whereas in Table 1 it is shown fully residing within the normal corridor), meaning that this measurement result (i.e., 7.15 μA) is, indeed, an indication to a problematic liver. Now, because, as shown in Table 2, the measurement result after the stimulation (i.e., 10 μA) is also shown falling outside, in this example, the normal corridor, a decisive conclusion is reached, according to which the diagnosed Liver is problematic.

TABLE 4
(modified corridor for the 1^{st} set of meas. results)

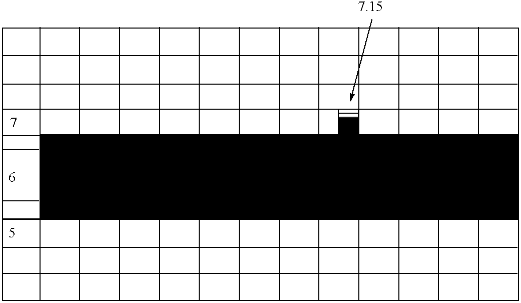

2. A modified corridor (I_{MC2}) is found for the second set of 24 measurement results (i.e., after the stimulation), as follows:

$${I}_{MC 2} = \{ \begin{matrix}( AV 2 / I 2 ) \times In ; & if AV 2 < I 2 \\ In ; & if I 2 \leq AV 2 \leq I 1 \\ ( AV 2 / I 1 ) \times In ; & AV 2 > I 1\end{matrix}$$

For the second set of 24 measurement results the condition I1<AV2<I2 is met (i.e., 7.40<AV2<10.60), and, therefore, the second corridor remains “as is” (i.e., unmodified), that is, I_{MC2}=In=2.50 μA.

Accordingly, with respect to the second set of measurement results, no changes are required with respect to the location of the upper and the lower borders of the corridor, and, therefore, Table 2 can be utilized “as is” (i.e., unchanged) for further analysis. That is, because, as shown in Table 2, the measurement result after the stimulation (i.e., 10 μA) is also shown falling outside the normal (i.e., in this case, the unmodified) corridor, a decisive conclusion is reached, according to which the diagnosed Liver is problematic.

Now, if desired, an average diagram may be plotted, upon which measurement results of the first and the second sets are superimposed on one another and compared. Before plotting the diagram, the measurement results of the first and the second sets are first normalized by calculating Δ1=|I_{MC1}−I_{MC2}| and, as follows:

The difference (Δ1=|I_{MCI}−I_{MC2}|) between the modified corridors is calculated:
Δ1=|I_{MC1}−I_{MC2}|=|2.00−2.50|=0.5

Because I_{MC1}<I_{MC2}, and conforming to the rules described hereinabove, the first original set of 24 measurement results (i.e., the results obtained prior to the stimulation) is modified by adding, to each one of these measurement results, a constant value (i.e., an offset value) equal to Δ/2=0.5/2=0.25 μA. Since the example refers only to one measurement, which relates, in this example, to a liver, only this measurement result is modified; i.e., only the exemplary measured value 7.15 μA (shown in Table 1) is initially modified to be 7.15+0.25=7.40 μA.

Then, the difference Δ2=|AV1−AV2|, between the corresponding isoelectric values (i.e., AV1=6.00 μA≠AV2=7.70 μA, see Table 4 and Table 2, respectively), is calculated to be 7.7−6.0=1.70 μA, and this difference is also added to each measurement result in Table 4. Accordingly, the previously calculated value 7.40 μA (the original value being 7.15 μA) is modified, a second time, to be 7.40+1.70=9.1 μA, which makes it exceeding the upper border of the modified corridor (i.e., 9.10>8.95), as shown in Table 5.

Of course, the order of calculation of Δ1=|I_{MC1}−I_{MC2}| and Δ2=AV1−AV2| can be reversed.

Then, the secondly modified measurement result (i.e., 9.1 pAu) and the corresponding unmodified result shown in Table 2 are superimposed on one another, the result being shown in Table 5, where reference numerals (1) and (2) denote the calculated, or modified, value, which relates to the measurement value before the stimulation, and reference numerals (2) and (3) denote the original, unmodified, measured result after the stimulation, and where reference numeral (2) denotes an overlapping area between the modified and unmodified value/result.

Finally, and referring to Table 5, because the original, unmodified, value (i.e., 10.00) of the measurement result after stimulation, and the original measurement result before stimulation and after being modified are both falling outside the corridor, a decisive conclusion is made, according to which the indication, in Table 2, of the presence of a disease in the liver is a true indication.

TABLE 5
(1^{st} set superimposed on 2^{nd} set, according to the invention)

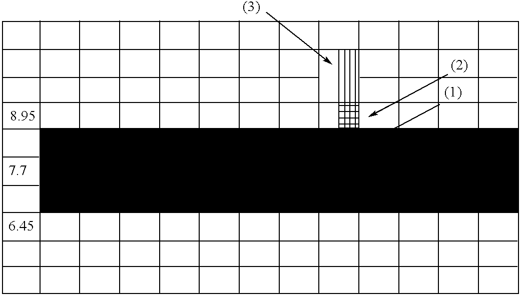

The above embodiments have been described by way of illustration only and it will be understood that the invention may be carried out with many variations, modifications and adaptations, without departing from its spirit or exceeding the scope of the claims.

1-19. (canceled)

20. An improved data analysis method, useful in a diagnostic method comprising selecting X biologically active points (BAPs), measuring the skin resistance at each one of said points relative to two fixed resistance values corresponding to a lower border and to an upper border of skin resistances, without stimulation and after stimulation, whereby to obtain two sets of measurement results, a first set for non-stimulated BAPs and a second for the same BAPs after being stimulated, for each set calculating the average resistance for said points as a first and a second isoelectric line, respectively, for which a first and a second normal corridors are defined, respectively, the method being:
(a) if the value of said first, and/or second isoelectric line, as the case may be, falls between the values of said lower border and said upper border, the corresponding normal corridor(s) remains unmodified, whereas if the value of said first, and/or second, isoelectric line falls below the value of said lower border, the corresponding normal corridor(s) is modified to have a narrower width, or, if the value of said first, and/or second, isoelectric line is greater than the value of said upper border, the corresponding normal corridor(s) is modified to have a larger width, the resulting modified width depending on the relationship existing between the value of a specific isoelectric line to the values of said lower and upper borders, and
(b) Diagnostic conclusions are reached based on the measurement results before and after the stimulation, and on the first and/or second normal corridors, whether modified or unmodified, whichever the case maybe.

21. A method according to claim 20, wherein the diagnostic conclusions comprises:
a. If a measurement result belonging to the first set falls outside the first modified, or unmodified, corridor, this result is considered a potential indicator of disease activity; and
b. If the corresponding result belonging to the second set also falls outside the second modified, or unmodified, corridor, the potential indicator is considered as a true indication of the presence of a disease; otherwise, said potential indicator is a false indicator and is, therefore, disregarded.

22. A method according to claim 21, wherein the modification of the width of the first and/or second normal corridors is performed by:
a. selecting one or more BAPs of interest and measuring the skin resistance at these points without stimulating them, whereby to obtain a first set of resistance results, said first set of results comprises a first group of measurement[i], where i=1, 2, , . . . , (up to i=X);
b. calculating the isoelectric line (AV1) relating to the first group of measurements measurement[i] (i =1, 2, 3, . . . , X):
$$AV 1 = ( {\sum }_{i = 1}^{x} measured [ i ] ) / X$$
where ‘X’ is the number of the measured BAPs;
c. modifying the normal corridor to obtain the corridor (I_{MC1}) relating to the first isoelectric line (AV1) using the following rules:
(c.1) if AV1<I2, then:
$${I}_{MC 1} = \frac{AV 1}{I 2} \times In ( in μ A )$$
where
I2 (the lower border of a corridor with respect to any isoelectric line) equals: I2=U/(R_{dev}+Rᵤₚ)−In(in μA);
‘U’ is the magnitude (in volts) of the voltage source;
‘R_{dev}’ is the intrinsic (i.e., “self”) resistance of the measurement apparatus (in kΩ); and
‘Rᵤₚ’ designates the highest value (in kΩ) of a range, the lower value of which is designated by ‘R_{low}’ (in kΩ), and the electrical resistance of BAPs normally (i.e., statistically) reside within the range {R_{low}, R^{up}};
(c.2) if I2≦AV1≦I1, then
I_{MCI}=In(in μA)
where I1 is the upper border of the isoelectric line and equals: I1=U/(R_{dev}+R_{low}) (in μA); and ‘In’ designates the span of the normal corridor (in μA); and
(c.3) if AV1>I1, then
$${I}_{MC 1} = \frac{AV 1}{I 1} \times In ( μ A )$$
d. applying stimulation to the BAPs used in step (a), and, essentially immediately thereafter, measuring the skin resistance at said points, whereby to obtain a second set of resistance results, said second set of results comprises a second group of measurement[i], where i=1′, 2′, 3′, . . . , (up to X BAPs, for which i=X′), where the indexes 1′ and 1 refer to the same BAP (1′—after stimulation, and 1—before stimulation), and so on;
e. calculating a second isoelectric line (AV2) relating to the second group of measurement[i], (i=1′, 2′, 3′, . . . , where 1 and ‘1’ refer to the same BAP, and so on):
$$AV 2 = ( {\sum }_{i = {1}^{′}}^{x} measure [ i ] ) / X$$
and
f. modifying the universal corridor to obtain the corridor (I_{MC2}) relating to the second isoelectric line (AV2) using the following rules:
(f.1) if AV2<I2, then:
$${I}_{MC 2} = \frac{AV 2}{I 2} \times In ( i n μ A )$$
(f.2) if I2≦AV2≦I1, then
I_{MC2}=In(μA)
(f.3) if AV2>I1, then
$${I}_{MC 2} = \frac{AV 2}{I 1} \times In ( μ A )$$

23. A method according to claim 22, wherein an average diagram is plotted, upon which the measurement results of the first and second sets are superimposed on one another and compared, after normalization and modification, the normalization being implemented by:
a. calculating a first difference Δ1=|I_{MC1}−I_{MC2}| and a second difference Δ2=|AV1−AV2|;
b. modifying the group of measurement results measurement[i] to obtain a modified set of measurement results measurement[i]_{modified}:
measurement[i]_{modified}=measurement[i]+Δ1/2+Δ2
where,
if I_{MC1}<I_{MC2}, then i=1, 2, 3, . . . , (up to X), or
if I_{MC1}>I_{MC2}, then i=1′, 2′, 3′, . . . , (up to X′),
it may occur that Δ1/2+Δ2=0;
and
c. superimposing the (first, or second) group of measurements, after modifying it in accordance with step (b) above, on the unmodified (second, or first) group of measurements, and plotting the results on a same diagram for comparison.

24. A method according to claim 22, wherein the number X of the BAPs is 24.

25. A method according to claim 23, wherein the number Xof the BAPs is 24.

26. A method according to claim 20 constituting a part of a diagnostic method.

27. A device adapted to measure electrical resistance at a number of BAPs relative to two fixed resistance values corresponding to a lower border and to an upper border of skin resistances, without stimulation and after stimulation, whereby to obtain two sets of measurement results, a first set for non-stimulated BAPs and a second for the same BAPs after being stimulated, for each set calculating the average resistance for said measurement results as a first and a second isoelectric line, respectively, for which a first and a second normal corridors are defined, respectively, the device being further adapted to perform data analysis as follows:
a. if the value of said first, and/or second isoelectric line, as the case may be, falls between the values of said lower border and said upper border, the corresponding normal corridor(s) remains unmodified, whereas if the value of said first, and/or second, isoelectric line falls below the value of said lower border, the corresponding normal corridor(s) is modified to have a narrower width, or, if the value of said first, and/or second, isoelectric line is greater than the value of said upper border, the corresponding normal corridor(s) is modified to have a larger width, the resulting modified width depending on the relationship existing between the value of a specific isoelectric line to the values of said lower and upper borders, and
b. Diagnostic conclusions are reached based on the measurement results before and after the stimulation, and on the first and/or second normal corridors, whether modified or unmodified, whichever the case maybe.

28. A device according to claim 27, where the diagnostic conclusions is reached as follows:
a. If a measurement result belonging to the first set falls outside the first modified, or unmodified, corridor, this result is considered a potential indicator of disease activity; and
b. If the corresponding result belonging to the second set also falls outside the second modified, or unmodified, corridor, the potential indicator is considered as a true indication of the presence of a disease; otherwise, said potential indicator is a false indicator and is, therefore, disregarded.

29. A device according to claim 28, wherein the modification of the width of the first and/or second normal corridors is performed by:
a. selecting one or more BAPs of interest and measuring the skin resistance at these points without stimulating them, whereby to obtain a first set of resistance results, said first set of results comprises a first group of measurement[i], where i=1, 2, 3, . . . , (up to i=X);
b. calculating the isoelectric line (AVI) relating to the first group of measurements measurement[i] (i=1, 2, 3, . . . , X):
$$AV 1 = ( {\sum }_{i = 1}^{x} measured [ i ] ) / X$$
where ‘X’ is the number of the measured BAPs;
c. modifying the normal corridor to obtain the corridor (I_{MC1}) relating to the first isoelectric line (AV1) using the following rules:
(c.1) if AV1<I2, then:
$${I}_{MC 1} = \frac{AV 1}{I 2} \times In ( i n μ A )$$
where
I2 (the lower border of a corridor with respect to any isoelectric line) equals: I2=U/(R_{dev}+Rᵤₚ)−In(in μA);
‘U’ is the magnitude (in volts) of the voltage source;
‘R_{dev}’ is the intrinsic (i.e., “self”) resistance of the measurement apparatus (in kΩ); and
‘Rᵤₚ’ designates the highest value (in kΩ) of a range, the lower value of which is designated by ‘R_{low}’ (in kΩ), and the electrical resistance of BAPs normally (i.e., statistically) reside within the range {R_{low}, Rᵤₚ};
(c.2) if I2≦AV1≦I1, then
I_{MC1}=In(in μA)
where I1 is the upper border of the isoelectric line and equals: I1=U/(R_{dev}+R_{low}) (in μA); and ‘In’ designates the span of the normal corridor (in μA); and
(c.3) if AV1>I1, then
$${I}_{MC 1} = \frac{AV 1}{I 1} \times In ( μ A )$$
d. applying stimulation to the BAPs used in step (a), and, essentially immediately thereafter, measuring the skin resistance at said points, whereby to obtain a second set of resistance results, said second set of results comprises a second group of measurement[i], where i=1′, 2′, 3′, . . . , (up to X BAPs, for which i=X′), where the indexes 1′ and 1 refer to the same BAP (1′—after stimulation, and 1—before stimulation), and so on;
e. calculating a second isoelectric line (AV2) relating to the second group of measurement[i], (i=1′, 2′, 3′, . . . , where 1 and ‘1’ refer to the same BAP, and so on):
$$AV 2 = ( {\sum }_{i = {1}^{′}}^{x} measure [ i ] ) / X$$
and
f. modifying the universal corridor to obtain the corridor (I_{MC2}) relating to the second isoelectric line (AV2) using the following rules:
(f.1) if AV2<I2, then:
$${I}_{MC 2} = \frac{AV 2}{I 2} \times In ( i n μ A )$$
(f.2) if I2≦AV2≦I1, then
I_{MC}2=In(μA)
(f.3) if AV2>I1, then
$${I}_{MC 2} = \frac{AV 2}{I 1} \times In ( μ A )$$

30. A device according to claim 29, further adapted to provide data from which an average diagram may be plotted, upon which the measurement results of the first and second sets are superimposed on one another and compared, after normalization and modification, the normalization being implemented by:
a. calculating a first difference Δ1=|I_{MC1}−I_{MC2}| and a second difference Δ2=|AV1−AV21;
b. modifying the group of measurement results measurement[i] to obtain a modified set of measurement results measurement[i]_{modified}:
measurement[i]_{modified}=measurement[i]+Δ1/2+Δ2
where,
if I_{MC1}<I_{MC2}, then i=1, 2, 3, . . . , (up to X), or
if I_{MC1}>I_{MC2}, then i=1″, 2″, 3″, . . . , (up to X″),
it may occur that Δ1/2+Δ2=0;
and
c. superimposing the (first, or second) group of measurements, after modifying it in accordance with step (b) above, on the unmodified (second, or first) group of measurements, and plotting the results on a same diagram for comparison.

31. A device according to claim 27, wherein the number of the BAPs is 24.

32. A device according to claim 28, wherein the number of BAPs is 24.

33. A device according to claim 27, further adapted to transmit data to a separate processing unit.

34. The device according to claim 27, wherein the same pressure is applied when measuring BAPs.

35. The device according to claim 34, wherein the pressure is substantially 0.5 Dj/cm².

36. A device according to claim 27, further adapted to measure each BAP as follows:
a. taking a plurality of sub-measurements within a short period of time;
b. calculating the range of the sub-measurements; and
c. repeating the sub-measurements until the range does not exceed a predetermined value.

37. A device according to claim 36, wherein 5 sub-measurements are taken with 0.02 seconds.

38. A device according to claim 36, wherein the predetermined value is 5%.

39. A device according to claim 27, further adapted to provide the stimulation.
